(19) United States
(12) Patent Application Publication
(10) Pub. No.: US 2010/0247653 A1
(43) Pub. Date: Sep. 30, 2010

(54) NANOPARTICLES CONTAINING NICOTINE AND/OR COTININE, DISPERSIONS, AND USE THEREOF

(76) Inventors: Hans Lautenschlager, Leichlingen (DE); Ilan Elias, Frankfurt am Main (DE)

Correspondence Address: CANTOR COLBURN, LLP, 20 Church Street, 22nd Floor, Hartford, CT, 06103, US

(21) Appl. No.: 12/297,155

(22) PCT Filed: Apr. 11, 2007
(86) PCT No.: PCT/EP2007/003217

§ 371 Date: Jun. 11, 2010

(30) Foreign Application Priority Data
Apr. 11, 2006 (DE) 10 2006 016 978.6

(51) Int. Cl. A61K 9/14; A61P 25/34; A61K 31/465; C07D 401/04; A61P 15/00; A61P 25/16; A61P 25/28

(52) U.S. Cl. 424/489; 514/343; 546/279.4; 546/278.4; 428/402; 977/773

(57) ABSTRACT

The present invention relates to nanoparticles containing nicotine and/or cotinine. The present invention further relates to dispersions containing these nanoparticles. The invention relates in particular to corresponding novel transdermal pharmaceuticals containing nicotine and cotinine in nanoparticulate form, and use thereof for smoking cessation.

### CLAIM FOR PRIORITY

This application is a National Stage filing under 37 USC 371 of International Patent Application No. PCT/EP2007/003217, filed Apr. 11, 2007. Applicants claim the benefit of the filing date of Apr. 11, 2006 to German Patent Application No. 10 2006 016 978.6 under provisions of 35 USC 119 and the International Convention for the protection of Industrial Property and the benefit of the filing date of the International Application under 35 USC 365, the entire contents of both of which are hereby incorporated by reference.

An embodiment of the present invention relates to nanoparticles containing nicotine and/or cotinine. An embodiment of the present invention further relates to dispersions containing these nanoparticles. An embodiment of the invention relates in particular to corresponding novel transdermal pharmaceuticals containing nicotine and cotinine in nanoparticulate form and use thereof for smoking cessation and for medicinal purposes, in particular for treatment of attention deficit hyperactivity disorder (ADHD), Parkinson's disease, Alzheimer's disease, or Binswanger's disease, and for the topical stimulation of external sexual organs, and corresponding treatment methods.

### BACKGROUND

Cigarette smoking results in the inhalation of nicotine as well as the intake of many undesirable substances which are sometimes toxic, carcinogenic, mutagenic, and teratogenic. Quitting smoking continues to be a concern of many smokers, but recidivism frequently occurs after periods of abstinence.

Therefore, there is a great need for agents that are suitable for assisting smokers in quitting smoking, wherein these agents should be able to deliver nicotine or cotinine in a controlled manner in the form of a monosubstance.

According to the prior art, aerosols or oral administration forms are frequently used, but these often result in undesirable side effects. Transdermal systems, in particular in the form of plasters, gels, salves, and films, have also become established. These are also referred to hereinafter as “base” or “bases.” Such systems must meet a number of requirements:
(1) The active substances nicotine and/or cotinine should be either finely dispersed or dissolved in the base.
(2) The active substances must be releasable from the base into the skin.
(3) The release should be controlled, i.e., should occur uniformly over a long period of time, to avoid undesirable concentration peaks. At the same time, however, penetration should be as rapid as possible and free of residues. Nevertheless, permeation and the subsequent action of the drug must be initiated briefly after administration.
(4) The ingredients of the base must be tolerable by the skin; i.e., they must not be irritating or sensitizing, and must not have an adverse effect on the regenerative property of the skin, in particular when they are used over a long period of time.
(5) The ingredients of the base should be as physiologically compatible as possible, and accordingly should not represent foreign bodies to the skin.

The transdermal systems known heretofore only partially meet requirements (1) through (5). For this reason, for the current commercially available transdermal systems undesirable changes to the skin frequently occur, primarily at the adhesion site. This results in reddening of the skin, swelling, itching, rashes, burning, and allergic symptoms.

Thus, for example, gels containing a high concentration of alcohol impair the barrier function of the skin by massive disruption of the so-called lipid double layers (bilayers), which are composed primarily of cholesterol, ceramides, and palmitic acid in a 1:1:1 ratio. This results not only in drying of the skin, but also in dermatosis after prolonged usage. This is true for high concentrations of ethanol as well as isopropyl alcohol, propylene glycol, and other mono- or polyhydric alcohols. In these systems alcohol is used as a solvent, and therefore is employed in high concentrations. Thus, to ensure that alcohol serves as a non-irritating, non-sensitizing, disinfectant agent, concentrations of less than or equal to 20 wt-% are desirable. In the known systems, however, as a result of these concentrations permeation is very unsatisfactory or totally absent, since it is specifically the disruption of the barrier function due to high alcohol concentrations that causes nicotine to be transported through the skin. For this reason, strictly aqueous solutions have proven to be completely ineffective, since the skin lipids, in particular for fatty skin, completely prevent penetration.

Similarly as for alcohols, all other solvent-based penetration accelerators such as dimethylsulfoxide (DMSO), for example, likewise disrupt the skin barrier, and also frequently have an unpleasant odor. For this reason they are less suitable for long-term use. O/W salves and creams containing emulsifiers also similarly damage the skin barrier, which is identifiable by an increased wash-out effect. Although this effect, which in particular results in intolerance reactions in atopic skin, is less pronounced for W/O salves and W/O creams, in this case the associated use of occlusive oils and waxes such as paraffin oil, Vaseline (petroleum jelly), and ozocerites in W/O formulations once again results in reduced regeneration capability of the skin. Furthermore, mineral oils and related substances significantly lower transepidermal water loss (TEWL), and the associated swelling of the skin results in impaired integrity of the skin barrier layers. The same is true for plasters and films as for mineral oils: the skin is sealed and the TEWL and regeneration capability of the skin are reduced. Therefore, none of these systems correspond to the physiological requirements of the skin.

For atopic skin, according to the prior art liposomal formulations using phosphatidylcholine (bilayers with an aqueous interior) have been proposed, but these have major disadvantages compared to nanoparticles:
(1) Adjuvant-free liposomal systems have only a very limited ability to store oils, generally 1% maximum. However, these oils are indispensable for optimal setting of a uniform release rate over a long period of time.
(2) The capability of liposomes for storing sterols is generally even lower than that for oils.
(3) The TEWL is increased by liposomes, so that the use of liposomes is not recommended for skin with barrier layer impairment.
(4) The release rate of nicotine from the horny layer of the epidermis is high and limited over time, since the retardation effect of the oils
   Therefore, a transdermal system would be desirable which meets requirements (1) through (5) described above and which does not have the described disadvantages of the previously known bases. This objective is particularly difficult to achieve, because on the one hand the active substances nicotine and cotinine should pass through the lipid double layers of the skin, and on the other hand the base must allow passage without impairing or destroying the lipid double layers (skin barrier). Consequently, the base should be chemically compatible with the skin components, must not physically damage the structure of the skin, and must be microbiologically acceptable. Water-containing systems, which are particularly susceptible, cannot be rendered microbiologically harmless using customary preservatives, since—as demonstrated, for example, in the list of permitted preservatives in the appendix of the European Cosmetics Directive—these substances may consistently have a sensitizing effect. The risk of sensitization is very high for transdermal systems, since the preservatives together with the nicotine or cotinine pass through the skin barrier. This is also true for fragrances, which likewise must be omitted in transdermal formulations. The objective may therefore be defined that the desired transdermal preparation must not contain preservatives, fragrances, emulsifiers, mineral oils, or substances that have an occlusive effect similar to mineral oils (for example, undifferentiated, long-chain silicones). This significantly complicates achievement of the object. It is further noted that penetration of the active substance into the horny epidermal layer of the skin, as stated above, must occur quickly, and permeation, i.e., transport, from the dead horny layer of the epidermis into the living skin layers therebelow must occur slowly and uniformly to likewise ensure rapid onset of activity and a persistent, continuous effect.

An embodiment of the invention, therefore, is to provide nicotine and/or cotinine in a form that may also be used in medicaments, and in which the corresponding agent is also improved with respect to the described disadvantages of the agents known from the prior art. This improvement should be ensured for various application forms, in particular topical administration. In the area of topical administration, improved absorption of the nicotine and/or its metabolite cotinine by the skin should be possible.

These advantages are achieved by means of the subject matter of the claims.

Surprisingly, it has been found that nanoparticles containing nicotine and/or cotinine are very well suited for significantly improving the properties of formulations for topical administration. For topical administration, the active substance (or active substances) is/are absorbed much better by the skin than when conventional formulations of these active substances are used.

Thus, the conditions for the desired transdermal system may surprisingly be realized by converting nicotine and/or its metabolite cotinine to the previously described nanoparticulate form.

The advantage of this nanoparticle dispersion, therefore, lies primarily in the very good pharmacological controllability. Because the newly developed transdermal system also functions practically without any films, plasters (patches), or other types of surface coverings, no undesirable changes in the skin are observed when it is used.

By use of the measures according to an embodiment of the invention, the nicotine and/or its metabolite cotinine also penetrate rapidly into the horny epidermal layer of the skin. Surprisingly, the active substance is slowly and uniformly released from the horny layer of the epidermis into the deeper skin layers. From the deeper skin layers the nicotine and/or its metabolite cotinine are able to pass into the bloodstream and thus be distributed through the entire body.

These advantages are achieved without the need for the administration form to contain compounds harmful to health that impair or destroy the lipid double layers of the skin (skin barrier). This advantage is realized due to the fact that the present dispersions contain only components which are physiologically tolerable by the skin, and which do not have an irritating or sensitizing effect and do not adversely affect the intrinsic regeneration of the skin. Surprisingly, the dispersions of the present invention are microbiologically stable.

This transdermal administration form according to an embodiment of the invention represents, in a manner of speaking, a virtual “intradermal patch system” which places a reservoir of nicotine and/or its metabolite cotinine in the skin, and allows nicotine and/or cotinine to be released from this reservoir for a specified period of time.

The described active substances nicotine and/or cotinine are known as such, and are commercially available from a number of sources. The referenced active substances have a relatively low molecular weight of 162.23 Dalton (nicotine) and 176.22 Dalton (cotinine).

In addition to the previously described active substances, the nanoparticles according to the invention may contain at least one physiologically acceptable fat and/or oil. Physiologically acceptable fats and/or oils include, among others, known plant oils used for skin care. Physiologically compatible triglycerides and esters, or mixtures thereof, are equivalent to the plant oils; solid esters and triglycerides may also be used, although the mixture is preferably liquid.

The preferred oils include, among others, castor oil, avocado oil, wheat germ oil, macadamia nut oil, sallow thorn oil, apricot kernel oil, almond oil, hemp oil, linseed oil, sesame oil, olive oil, soybean oil, sunflower oil, palm kernel oil, coconut oil, maize oil, palm oil, rapeseed oil, safflower oil, jojoba oil, peanut oil, shea butter, and cocoa butter. The oils referenced above may be used individually or as a mixture. In addition to natural oils, hydrogenated oils, often referred to as hardened oils, may be used.

The above-referenced oils frequently include esters of palmitic acid, stearic acid, oleic acid, ricinoleic acid, 11-hydroxypalmitic acid, or 12-hydroxystearic acid, and/or of myristic acid, caproic acid, caprylic acid (MCT component), capric acid (MCT component), lauric acid, isostearic acid, linoleic acid, and adipic acid. Esters of fatty acids containing 6 to 12 carbon atoms may also be used. The alcohol components of the esters are likewise known, wherein monohydric as well as polyhydric alcohols are suitable, for example ethanol, methanol, isopropyl alcohol, cetyl alcohol, glycerin, oleyl alcohol, octanol, isobutanol, and butanol. Examples of triglycerides and esters include isopropyl palmitate, isopropyl stearate, isopropyl myristate, triolein, oleic acid ethyl ester, isostearic acid ester, palmitic acid cetyl ester, saturated medium chain triglycerides (MCT), octyl stearate, octyldodecylstearoyl stearate, mono-, di-, tri-, and polyglycerides of ricinoleic, 12-hydroxystearic, 11-hydroxypalmitic, and oleic acids, and ricinoleic acid octyldodecyl ester, 12-hydroxystearic acid octyl ester, linoleic acid methyl and ethyl esters, and linoleic acid mono-, di-, and triglycerides.

Preferred nanoparticles according to an embodiment of the invention may also contain at least one sterol. Preferred sterols include in particular cholesterol, β-sitosterol, stigmasterol and/or campesterol, ergosterol, lanosterol, fucosterol, brassicasterol, and fungisterol. These compounds may be used individually or as a mixture in pure form, enriched, or as a natural component of waxes. “Enriched” refers to sterols containing, for example, unsaponifiables of avocado oil or other fatty oils. Sterols are also components of shea butter, cocoa butter, lanolin, and/or lanolin alcohols. The natural oils and fats used frequently contain sterols in sufficient quantities.

Preferred nanoparticles may also contain at least one phosphatidylcholine in native and/or hydrogenated form.

The nanoparticles preferably have a size in the range of 50 to 500 nm, most preferably in the range of 60 to 150 nm. Depending on the intended use of the medicament, one skilled in the art produces the nanoparticles in a suitable manner known as such.

For example, the conditions for the desired transdermal system may be achieved by dissolving the nicotine, which has a relatively low molecular weight (162.23 Dalton), or its metabolite cotinine (176.22 Dalton) in a physiologically acceptable fat and/or oil, preferably a plant oil used for skin care, which optimally also contains sterols which are structurally similar to cholesterol present in the skin. Physiologically compatible triglycerides and esters or mixtures thereof are equivalent to the plant oils; solid esters and triglycerides may also be used, wherein the mixture preferably has an oily consistency. If necessary, sterols may be added to the nicotine-oil mixture or the cotinine-oil mixture if they are not already contained in the natural plant oils and fats. Sterols, the same as cholesterol, have an effect that strengthens the skin barrier. The nicotine-oil solution or cotinine-oil solution, together with phosphatidylcholine and an appropriate quantity of water or diluted alcohol, is subjected to high-pressure homogenization. This results in liquid nanoparticles that are characterized by a particularly small size of approximately 50-500 nm (depending on the concentrations of the individual components). According to this production procedure, the surface of the nanoparticle is composed of phosphatidylcholine and optionally sterols in part, whereas the core of the nanoparticle contains the oil together with the dissolved nicotine or cotinine. Therefore, this particle is similar to plant or animal cells in size, and in its shell contains phosphatidylcholine, the most important material of the cell membranes in plant and animal cells. The nanoparticles differ from living cells in that in the former the phosphatidylcholine is provided in monolayers, whereas in the cell membranes the phosphatidylcholine forms bilayers, where it encloses an aqueous interior. On account of their composition, upon penetration into the horny layer of the epidermis the nanoparticles produced in this manner integrate into the lipid double layers of the skin barrier without impairment, and at that location release the active substance nicotine or cotinine in a very controlled manner into the deeper living skin layers (permeation). Measurements demonstrate that the individual TEWL of the skin remains constant or decreases only slightly. The reduction of the TEWL, if it occurs at all, is a temporary effect which is eliminated by the partially enzymatic decomposition of the infiltrated triglycerides into di- and monoglycerides and glycerin after several hours. The same applies for the use of physiologically compatible esters. This regulation corresponds to the functional mechanism of the natural homeostasis of the skin.

Therefore, an embodiment of the invention further relates to dispersions that contain the nanoparticles.

In addition to the previously described components, these dispersions contain in particular water and/or alcohols. The proportion of water in the dispersion is preferably in the range of 30 wt-% to 95 wt-%. Preferred alcohols preferably contain 2 to 6 carbon atoms, wherein these alcohols may contain 1, 2, 3, or more hydroxyl groups. Examples of preferred alcohols include ethyl alcohol, propylene glycol, butylene glycol, pentylene glycol, hexylene glycol, glycerin, isopropyl alcohol, and sorbitol, wherein these alcohols may be used individually or as a mixture. The dispersions most preferably contain water and at least one alcohol. The proportion of the alcohol (or alcohols) is preferably 5 wt-% to 25 wt-%, most preferably 10 wt-% to 20 wt-%.

Preferred dispersions contain
(1) 0.01 to 10 wt-%, 0.5 to 2 wt-% nicotine and/or cotinine,
(2) 2 to 40 wt-%, 5 to 30 wt-% physiologically acceptable fats and/or oils,
(3) 0.01 to 5 wt-% sterol, and
(4) 0.5 to 10 wt-%, preferably 2 to 5 wt-%, phosphatidylcholine in native and/or hydrogenated form,
(5) 5 to 25 wt-% alcohol, and
(6) 30 to 95 wt-% water, in each case relative to the total weight of the dispersion.

Surprisingly, it has also been shown that the novel transdermal system requires no additional (auxiliary) substances to ensure the previously mentioned conditions (1) through (5). The conditions of chemical and physical compatibility are likewise met. The nanoparticle dispersion is microbiologically stable when a final alcohol content between 10% and 20% is maintained. Thus, the dispersion is also free of any sensitizing or irritating substances or substance concentrations. The novel nanoparticles may be dosed in a very simple manner by applying them to the skin using a pipette or by means of an ampule. The dispersion according to the invention is absorbed, without residues, by the horny layer of the epidermis immediately after distribution. Films, plasters, or other types of surface coverings are therefore unnecessary. The subsequent permeation occurs uniformly from the horny layer of the epidermis through the skin layers therebelow over a long period of time, which may be shortened or lengthened as desired by virtue of the type and/or quantity of oils and/or fats used and the sterols contained therein.

The nicotine nanoparticles may be applied to healthy skin as well as to skin with barrier impairment, since phosphatidylcholine promotes long-term formation of ceramide I, the most important barrier material in the skin, as a result of its linoleic acid content. Therefore, the nanoparticle dispersion is also particularly well suited for atopic skin.

Accordingly, one embodiment of the nicotine or cotinine nanoparticle dispersion may be primarily composed of components (1) through (5):
(1) Nicotine and/or cotinine.
(2) Plant oils or equivalent triglycerides or triglyceride mixtures and physiologically compatible esters: (a) fatty oils, for example avocado oil, wheat germ oil, macadamia nut oil, sallow thorn oil, apricot kernel oil, almond oil, hemp oil, linseed oil, sesame oil, olive oil, soybean oil, sunflower oil, and peanut oil in natural and hydrogenated form; (b) triglycerides and esters, for example isopropyl palmitate, isopropyl stearate, isopropyl myristate, triolein, oleic acid ethyl ester, isostearic acid ester, palmitic acid cetyl ester, saturated medium chain triglycerides (MCT), octyl stearate, octyldodecylstearoyl stearate, mono-, di-, tri-, and polyglycerides of ricinoleic, 12-hydroxystearic, 11-hydroxypalmitic, and oleic acids, and castor oil, hardened castor oil, ricinoleic acid octyldodecyl ester, 12-hydroxystearic acid octyl ester, linoleic acid methyl and ethyl esters, and linoleic acid mono-, di-, and triglycerides.
(3) Sterols in pure or enriched form, or as a natural component of waxes: (a) pure sterols, for example cholesterol and stigmasterol; (b) enriched, for example the unsaponifiables of avocado oil or other fatty oils; (c) as components of shea butter, cocoa butter, lanolin, or lanolin alcohols.
(4) Phosphatidylcholine in native and hydrogenated form.
(5) Alcohols, which are understood to mean alcohols in the form of their mono- to hexahydric representatives, containing 2-6 carbon atoms and primary or secondary alcoholic hydroxyl groups, wherein the carbon chain may be linear, branched, or ring-shaped. Examples include ethyl alcohol (alcohol), propylene glycol, butylene glycol, pentylene glycol, hexylene glycol, glycerin, sorbitol, or mixtures thereof
(6) Water.

Besides the main components, in individual cases additional skin care substances may be added. These are understood to be substances tolerable to the skin, such as jojoba oil, vitamins, D-panthenol, urea, etc., but whose concentrations are insignificant for the actual transdermal effect of the nicotine or cotinine. If, for example, oils are used which are susceptible to oxidation, addition of antioxidants such as vitamin E and vitamin C and derivatives thereof such as acetates, palmitates, and phosphates may be meaningful if these or similar substances are not already contained in the fatty oils. Avocado oil, for example, contains considerable quantities of vitamin E.

The nicotine nanoparticle dispersion or cotinine nanoparticle dispersion according to an embodiment of the invention may be used as such in the pure form, or in combination with suitable barrier creams having a “derma membrane structure,” familiar to one skilled in the art as the abbreviation DMS, and, the same as the barrier layers of the horny layer of the epidermis, containing so-called bilayers.

The nicotine or cotinine nanoparticle dispersions described by way of example may be applied on the arm, such as at the bend of the arm, in the arm pit, in the pubic region, on the abdomen or back, the feet, or any other given locations (skin and mucous membranes) on the entire body. With regard to its fat content, the skin may be low or high in fat, and with regard to its moisture content may be dry to moist.

The composition of the novel nicotine or cotinine nanoparticle dispersion is explained in the following examples.

### EXAMPLES

The sizes of the nanoparticles were measured by laser light scattering (photon correlation spectroscopy).

### Example 1

0.1 g nicotine was dissolved in 12 g avocado oil (containing natural sterols) and mixed with a solution of 7 g phosphatidylcholine in 18 g alcohol. The mixture together with 62.9 g water was repeatedly subjected to high-pressure homogenization until the resulting nanoparticles had attained an average size of 150 nm.

### Example 2

A mixture of 19 g medium chain triglycerides, 9.5 g phosphatidylcholine, 16 g alcohol, 0.5 g stigmasterol, and 1 g cotinine was heated to 50° C., and after addition of 54 g water was repeatedly subjected to high-pressure homogenization until nanoparticles having an average size of 125 nm were obtained.

### Example 3

A mixture of 10 g medium chain triglycerides, 5 g avocado oil (containing natural sterols), 4 g shea butter (containing natural sterols), 8 g phosphatidylcholine, 6 g pentylene glycol, 4 g propylene glycol, 7.5 g glycerin, and 0.5 g nicotine was heated to 60° C., and after addition of 55 g water was repeatedly subjected to high-pressure homogenization until nanoparticles having an average size of 110 nm were obtained.

### Example 4

1 g nicotine was dissolved in 12 g avocado oil (containing natural sterols) and mixed with a solution of 7 g phosphatidylcholine in 18 g alcohol. The mixture together with 62 g water was repeatedly subjected to high-pressure homogenization until the resulting nanoparticles had attained an average size of 130 nm.

### Example 5

0.5 g nicotine was combined with 0.2 g Avocadin (sterol-containing avocado oil extract), 15.8 g alcohol, 0.02 g urea, 12 g olive oil, 0.2 g tocopherol acetate, 6 g phosphatidylcholine, and 0.2% hydrogenated phosphatidylcholine and 65.08 g water, heated to 60° C., and subjected to high-pressure homogenization until the resulting nanoparticles had attained an average size of 150 nm.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. Nanoparticles containing nicotine and/or cotinine.

2. The nanoparticles according to claim 1, wherein the nanoparticles have a size in the range of 50 to 500 nm.

3. The nanoparticles according to claim 1, wherein the nanoparticles contain physiologically acceptable fats and/or oils.

4. The nanoparticles according to claim 3, wherein the physiologically acceptable fats and/or oils contain esters of fatty acids containing 6 to 12 carbon atoms.

5. The nanoparticles according to claim 3, wherein the physiologically acceptable fats and/or oils contain esters of palmitic acid, stearic acid, oleic acid, ricinoleic acid, 11-hydroxypalmitic acid, 12-hydroxystearic acid, and/or myristic acid.

6. The nanoparticles according to claim 1, wherein the nanoparticles contain at least one sterol.

7. The nanoparticles according to claim 1, wherein the nanoparticles contain at least one phosphatidylcholine in native and/or hydrogenated form.

8. A dispersion containing nanoparticles according to claim 1.

9. The dispersion according to claim 1, wherein the dispersion contains water and alcohol.

10. The dispersion according to claim 8, wherein the dispersion contains
0.01 to 10 wt-% nicotine and/or cotinine,
2 to 40 wt-% physiologically acceptable fats and/or oils,
0.01 to 5 wt-% sterol,
0.5 to 10 wt-% phosphatidylcholine in native and/or hydrogenated form,
5 to 25 wt-% alcohol, and
30 to 95 wt-% water,
in each case relative to the total weight of the dispersion.

11. The dispersion according to claim 9, wherein the alcohol contains 2 to 6 carbon atoms.

12. The dispersion according to claim 8, wherein the dispersion is free of sensitizing or irritating substances.

13. A pharmaceutical containing nanoparticles according to claim 1, or containing at least one dispersion according to claim 8.

14. The pharmaceutical according to claim 13, which is suitable for topical administration.

15. The pharmaceutical according to claim 14 in the form of a salve, cream, lotion, gel, or suppository.

16. Use of nanoparticles according to claim 1 for withdrawal from tobacco or at least one dispersion according to claim 8, for withdrawal from tobacco.

17. Use according to claim 16 for smoking cessation.

18. Use of nanoparticles according to claim 1 for medicinal purposes or at least one dispersion according to claim 8, for medicinal purposes.

19. Use according to claim 18 for treatment of attention deficit hyperactivity disorder (ADHD), Parkinson's disease, Alzheimer's disease, or Binswanger's disease.

20. Use according to claim 18 for the topical stimulation of external sexual organs.
